(19) United States
(12) Patent Application Publication
(10) Pub. No.: US 2026/0232431 A1
(43) Pub. Date: Aug. 13, 2026

(54) INTRAOCULAR LENS INJECTOR

(71) Applicant: Hoya Medical Singapore Pte. Ltd., Singapore (SG)

(72) Inventor: Kazunori KUDO, Saku-shi (JP)

(21) Appl. No.: 19/376,691

(22) Filed: Oct. 31, 2025

Related U.S. Application Data
(63) Continuation of application No. 17/435,762, filed on Dec. 23, 2019, now Pat. No. 12,478,467.

(30) Foreign Application Priority Data
Mar. 13, 2019 (JP) 2019-045673

(51) Int. Cl. A61F 2/16; A61F 9/007

(52) U.S. Cl. CPC A61F 2/167; A61F 2/1678; A61F 9/00736; A61F 2002/1681

(57) ABSTRACT

There is provided an intraocular lens injector for improving accuracy of tacking, which is an intraocular lens injector 1 for injecting an intraocular lens 4 into an eye, the intraocular lens including an optical portion 41 having an optical function and support portions 42a and 42b extending from the optical portion 41, including: a protrusion 20 for bending a forward support portion 42a while abutting against the forward support portion 42a, the forward support portion being one of the support portions 42a and 42b and being disposed on the front side in an advancing direction of the intraocular lens, when making the intraocular lens 4 advance along an inner wall of a hollow body in which the intraocular lens 4 is set and thereby releasing the intraocular lens 4 from the hollow body in a state in which the intraocular lens 4 is folded with an inner surface of the optical portion 41 facing inward, wherein the protrusion 20 has a retainer that temporarily suppresses progress of bending of the forward support portion 42a by retaining the forward support portion 42a, when the intraocular lens 4 is being folded.

### CROSS-REFERENCE TO RELATED APPLICATIONS

This application a continuation of U.S. application Ser. No. 17/435,762 , filed Sep. 2, 2021, which is the U.S. National Stage of PCT App. Ser. No. PCT/JP2019/050238, filed Dec. 23, 2019, each of which is incorporated herein by reference in its entirety.

### TECHNICAL FIELD

The present invention relates to an intraocular lens injector.

### BACKGROUND ART

In cataract surgery, removal of an opacified crystalline lens by phacoemulsification and implantation of an intraocular lens into an eye after the removal of the crystalline lens are widely performed. Nowadays, a soft intraocular lens made of a soft material such as silicone elastomer is injected into an eye using an intraocular lens injector.

When injecting a soft intraocular lens into an eye, it is possible to fold the intraocular lens, and therefore, cataract surgery can be performed with a smaller corneal incision.

A method for folding an intraocular lens is known in which the intraocular lens is folded within an intraocular lens injector (for example, Patent Document 1 to the applicant of the present invention). According to the method disclosed in Patent Document 1, a protrusion is provided to a case, and when the intraocular lens injector and the case are fitted to each other, the protrusion of the case protrudes from a protrusion insertion hole formed in the intraocular lens injector (paragraph 0038 and FIGS. 3 to 6, etc.).

In this state, the intraocular lens including a substantially disk-shaped optical portion having an optical function and a pair of support portions extending from the optical portion is advanced toward a discharge hole of a nozzle portion. As the intraocular lens is advanced, a leading end of a forward support portion of the intraocular lens abuts against the protrusion and can thus be bent rearward (paragraph 0041 and FIGS. 6 and 7, etc.).

### CITATION LIST

### Patent Documents

Patent Document 1: Japanese Patent No. 5254669

### SUMMARY OF INVENTION

### Technical Problem

With the method disclosed in Patent Document 1, the forward support portion of the intraocular lens can be bent rearward. On the other hand, as a result of examination by the inventor of the present invention, it was found that this method can cause the following phenomenon.

With the method disclosed in Patent Document 1, due to the advancement of the intraocular lens, the forward support portion of the intraocular lens is bent rearward, and the optical portion of the intraocular lens is folded. It is ideal that a surface (hereinafter referred to as an “inner surface”) that faces inward when the optical portion is folded wraps around the forward support portion. Such an ideally folded state is also referred to as “tacking”.

On the other hand, as a result of examination by the inventor of the present invention, it was found that, when the intraocular lens is advancing, there is a risk that the forward support portion will enter between an outer surface of the optical portion that is the opposite surface to the inner surface and an inner wall of the intraocular lens injector.

If the forward support portion enters between the outer surface of the optical portion and the inner wall of the intraocular lens injector, an operating surgeon has to undo the distorted bend of the forward support portion after releasing the intraocular lens, or in other words, in an eye of a patient. In that case, a large burden is placed on the operating surgeon.

It is a technical object of the present invention to provide an intraocular lens injector that improves the accuracy of tacking.

### Solution to Problem

The inventor of the present invention has conducted an in-depth study to achieve the above object. In the study, the inventor of the present invention focused on the movement of the forward support portion when abutting against the protrusion disclosed in Patent Document 1. As a result, it became clear that, with the protrusion disclosed in Patent Document 1, during the advancement of the entire intraocular lens in a forward direction, the timing of movement of the leading end of the forward support portion to the vicinity of the inner wall of the intraocular lens injector as a result of the forward support portion continuing to bend rearward and the timing of advancement of a peripheral edge of the optical portion of the folded intraocular lens tend to be extremely close to each other, or the leading end of the forward support portion tends to move to the inner wall in advance.

Thus, in order to intentionally shift these timings from each other, the inventor of the present invention conceived a technical idea of temporarily retaining the leading end of the forward support portion bending rearward during the advancement of the entire intraocular lens, thereby suppressing the bending of the forward support portion and delaying the timing of the movement of the leading end of the forward support portion to the vicinity of the inner wall of the intraocular lens injector.

Configurations obtained based on the above-described findings are as follows.

A first aspect of the present invention is
an intraocular lens injector for injecting an intraocular lens into an eye, the intraocular lens including an optical portion having an optical function and support portions extending from the optical portion, the intraocular lens injector including:
a protrusion for bending a forward support portion while abutting against the forward support portion, the forward support portion being one of the support portions and being disposed on the front side in an advancing direction of the intraocular lens, when making the intraocular lens advance along an inner wall of a hollow body in which the intraocular lens is set and thereby releasing the intraocular lens from the hollow body in a state in which the intraocular lens is folded with an inner surface of the optical portion facing inward,
wherein the protrusion has a retainer that temporarily suppresses progress of bending of the forward support portion by retaining the forward support portion, when the intraocular lens is being folded.

A second aspect of the present invention is the invention according to the first aspect, including:
the hollow body which is an injector main body;
a lens setting portion provided inside the hollow body;
an advancing member for advancing the intraocular lens disposed in the lens setting portion;
the protrusion which is disposed forward of the lens setting portion and protrudes from the inner wall of the hollow body; and
an injection tube disposed at a front end portion of the hollow body, the injection tube having a hollow injection tube body and a tubular nozzle portion,
the protrusion having a first abutting portion for bending the forward support portion while abutting against the forward support portion when the intraocular lens is being advanced by the advancing member, and the retainer disposed forward of the first abutting portion, and
the retainer having a support receiving portion for supporting the forward support portion after the forward support portion is separated from the first abutting portion, and a second abutting portion which is disposed forward of the support receiving portion and abuts against the forward support portion while the forward support portion is supported by the support receiving portion.

A third aspect of the present invention is the invention according to the second aspect,
wherein a part between the first abutting portion and the retainer are chamfered.

A fourth aspect of the present invention is
an intraocular lens injector for injecting an intraocular lens into an eye, the intraocular lens including an optical portion and support portions extending from the optical portion, the intraocular lens injector including:
a hollow body having a lens setting portion on which the intraocular lens is set, with an optical axis direction of the intraocular lens serving as a vertical direction; and
a protrusion protruding from an inner wall of the hollow body, and having at least one step directed upward when viewed from the lens setting portion in a forward direction, and a wall disposed forward of the step.

A fifth aspect of the present invention is the invention according to the fourth aspect,
wherein the protrusion protrudes from bottom to top of the inner wall of the hollow body, and has at least two steps directed upward when viewed from the lens setting portion in the forward direction.

A sixth aspect of the present invention is the invention according to any one of the first to fifth aspects,
wherein a slope is disposed at a rearmost side of the protrusion.

A seventh aspect of the present invention is the invention according to any one of the first to sixth aspects, further including:
a case configured to be fitted to the hollow body,
wherein the protrusion is provided to the case and a hole is formed in the hollow body so that the protrusion and the hole can be fitted to each other.

An eighth aspect of the present invention is the invention according to any one of the first to seventh aspects, further including:
the intraocular lens,
wherein the optical portion and a base end of each support portion in the intraocular lens are made of a soft material, and
the optical portion of the intraocular lens has an effective optical portion exhibiting an optical function, and a flat peripheral portion disposed around the effective optical portion.

### Advantageous Effects of Invention

According to the present invention, it is possible to provide an intraocular lens injector that improves the accuracy of tacking.

### BRIEF DESCRIPTION OF DRAWINGS

FIG. 1 is a perspective view showing an example of an external configuration of an intraocular lens injector according to an embodiment of the present invention.

FIG. 2 is a perspective view showing a structure and an arrangement of a leading end portion of an injector main body according to the embodiment of the present invention.

FIG. 3 is a perspective view of a case of the intraocular lens injector according to the embodiment of the present invention.

FIG. 4 is a perspective view showing a state in which the intraocular lens injector according to the embodiment of the present invention is attached to the case.

FIG. 5 is a perspective view showing a state of a protrusion and its vicinity in the intraocular lens injector according to the embodiment of the present invention.

FIG. 6 is a side view showing a state of the protrusion and its vicinity in the intraocular lens injector according to the embodiment of the present invention, when viewed in a direction from Y1 toward Y2.

FIG. 7 is a schematic cross-sectional view showing movements of a forward support portion (when viewed in cross section) relative to the protrusion in the intraocular lens injector according to the embodiment of the present invention.

FIG. 8 is a schematic side view showing a state of a protrusion and its vicinity in an intraocular lens injector according to a variation (first variation) of the present invention, when viewed in the direction from Y1 toward Y2.

FIG. 9 is a schematic side view showing a state of a protrusion and its vicinity in an intraocular lens injector according to a variation (second variation) of the present invention, when viewed in the direction from Y1 toward Y2.

FIG. 10 is a schematic side view showing a state of a protrusion and its vicinity in an intraocular lens injector according to a variation (third variation) of the present invention, when viewed in the direction from Y1 toward Y2.

### DESCRIPTION OF EMBODIMENTS

Hereinafter, an embodiment of the present invention will be described in detail with reference to the drawings. In the present specification, “to” between numerical values means being greater than or equal to a predetermined numerical value and less than or equal to a predetermined numerical value.

In the present embodiment, in order to describe relative positional relationships between various portions of an intraocular lens injector, directions in which they move, and the like, one of the directions along the X-axis is referred to as a “direction X1” and the other direction as a “direction X2”, one of the directions along the Y-axis is referred to as a “direction Y1” and the other direction as a “direction Y2”, and one of the directions along the Z-axis is referred to as a “direction Z1” and the other direction as a “direction Z2”. Moreover, the direction X1 is defined as a leading end side (forward, a lens advancing direction), the direction X2 is defined as a rear end side (rearward), the direction Y1 is defined as a left side (leftward), the direction Y2 is defined as a right side (rightward), the direction Z1 is defined as a top side (upward, a thickness direction and an optical axis direction of an intraocular lens when it is disposed), and the direction Z2 is defined as a bottom side (downward). Of these directions, the directions X1 and X2 correspond to a length direction of an intraocular lens injector 1, the directions Y1 and Y2 correspond to a width direction of the intraocular lens injector, and the directions Z1 and Z2 correspond to a height direction of the intraocular lens injector 1.

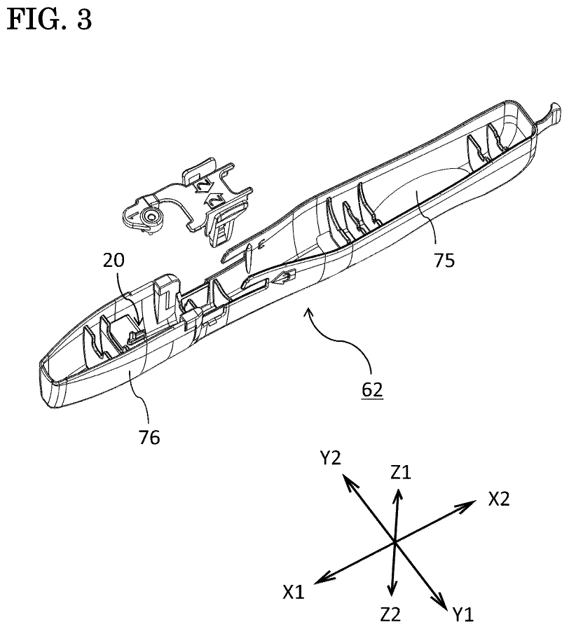

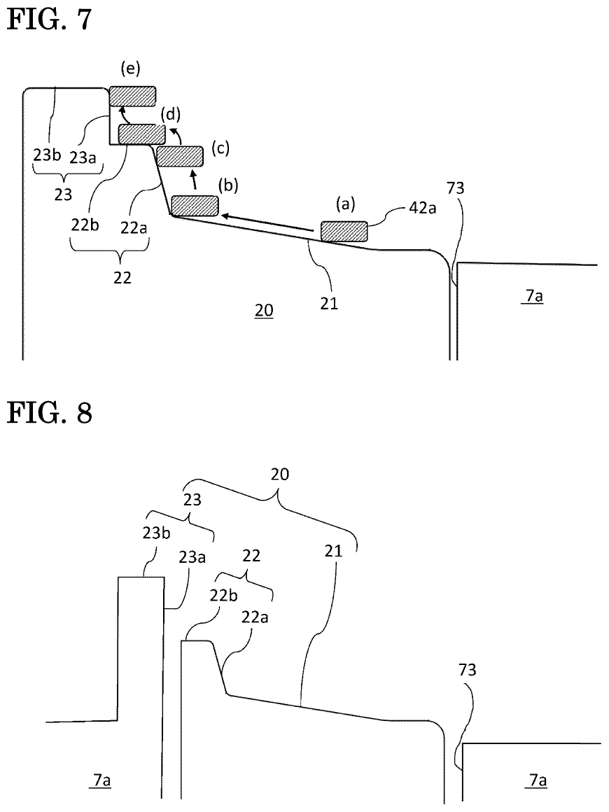

Note that it should be mentioned in advance that main characterizing portions of the present embodiment correspond to aspects of a protrusion 21 (FIGS. 3 to 6 etc. of the first embodiment of Patent Document 1), a protrusion 52 (FIG. 7 etc. of the second embodiment of Patent Document 1), and a protrusion 80 (FIGS, 8, 10, and 11, etc. of the third embodiment of Patent Document 1) disclosed in Patent Document 1. For this reason, descriptions of portions other than those related to a protrusion for bending a forward support portion of the intraocular lens injector are simplified.

Configurations of known intraocular lens injectors may be adopted for portions other than those related to the protrusion for bending the forward support portion. For example, configurations disclosed in WO 2018/003854 etc. to the applicant of the present invention may be adopted. For content that is not described below, configurations of known intraocular lens injectors (e.g., configurations disclosed in WO 2018/003854) are adopted, and the description of WO 2018/003854 is incorporated in the present specification in its entirety.

### Configuration of Intraocular Lens Injector

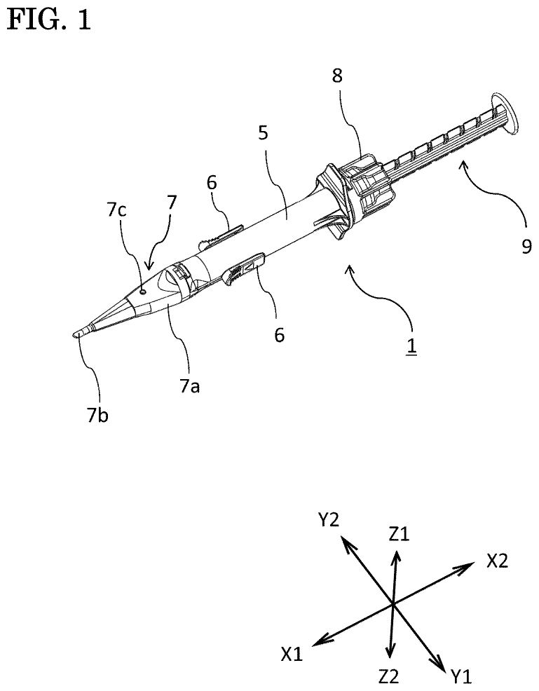

FIG. 1 is a perspective view showing an example of an external configuration of an intraocular lens injector according to an embodiment of the present invention.

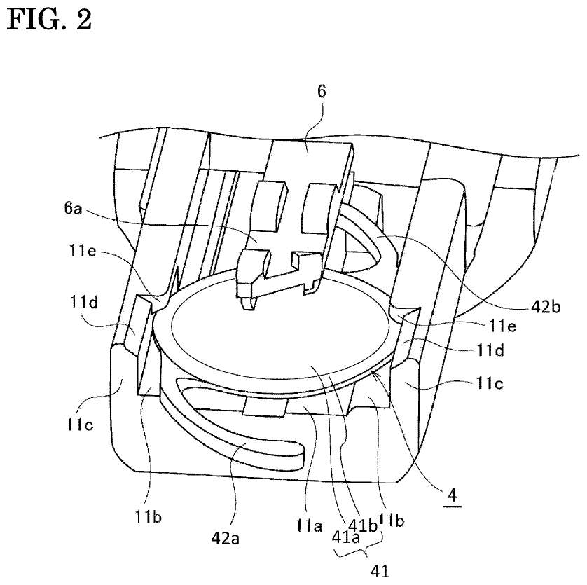

FIG. 2 is a perspective view showing a structure and an arrangement of a leading end portion of an injector main body according to the embodiment of the present invention.

Note that FIGS. 1 and 2 of the present application are similar to FIGS. 1 and 6 of WO 2018/003854 in terms of configurations other than an intraocular lens 4. For this reason, among the reference signs shown in FIG. 2 of the present application, reference signs that are not described in the present specification are as described in WO 2018/003854.

The intraocular lens injector 1 is used when injecting an intraocular lens into an eye. In the present embodiment, as an example of the intraocular lens, there is provided a one-piece type intraocular lens 4 (see FIG. 2) made of a soft material such as silicone elastomer or soft acrylic, the intraocular lens 4 including an optical portion 41 with a circular effective optical portion 41a that performs an optical function and a peripheral portion 41b thereof, and two support portions 42a and 42b that extend outward in a curved manner from two positions on an outer circumferential portion of the optical portion 41. Reference sign 42a denotes a forward support portion that is disposed on a front side when the intraocular lens is set on a lens setting portion 11, and reference sign 42b denotes a rearward support portion that is disposed on a rear side.

A case in which the optical portion 41 of the intraocular lens 4 and base ends of the respective support portions 42a and 42b connected to the optical portion 41 are made of a soft material is given as an example. This configuration facilitates folding of the intraocular lens 4. Note that distal ends of the respective support portions 42a and 42b may be similarly made of a soft material, or may be made of a hard material (e.g., polyethylene, PMMA, etc.).

Note that the effective optical portion 41a of the optical portion 41 has a shape that realizes the optical function, the peripheral portion 41b is formed in a ring shape around the effective optical portion 41a, and the ring is in a flat plate form. It goes without saying that a configuration other than this may also be adopted. For example, a configuration may be adopted in which the optical portion 41 is composed only of the effective optical portion 41a. Also, a configuration may be adopted in which the peripheral portion 41b is formed to be continuous with the effective optical portion 41a, or in other words, the peripheral portion 41b and the effective optical portion 41a are smoothly connected to each other and form a continuous surface together.

The intraocular lens injector 1 includes an injector main body 5, sliders 6, an injection tube 7, a rotary member 8, a plunger 9, and a rod (disposed inside and hidden by the intraocular lens injector 1 in FIG. 1 and therefore not shown). Preferably, these constituent elements are each constituted by a resin molded product.

The injector main body 5 and the injection tube 7 have hollow structures and are connected to each other to thereby form a hollow body.

The sliders 6 are attached to the injector main body 5.

The injection tube 7 is disposed so as to be in communication with a leading end portion of the injector main body 5. The injection tube 7 and the leading end portion of the injector main body 5 may be integrally molded, or may be separately molded and combined by attaching the injection tube 7 to the leading end portion. The injection tube 7 has a hollow injection tube body 7a and a narrow tubular nozzle portion 7b. In this case, the lens setting portion 11 of the injector main body 5, together with the intraocular lens 4 set thereon, is housed and disposed in the injection tube body 7a of the injection tube 7. An injection hole 7c is formed in an upper surface of the injection tube body 7a. The injection hole 7c is a hole through which a viscoelastic substance (for example, sodium hyaluronate etc.) is injected. The viscoelastic substance injected from the injection hole 7c is supplied to the intraocular lens 4 that is set on the lens setting portion 11.

The rotary member 8 is rotatably connected to a rear end portion of the injector main body 5.

The plunger 9 is disposed coaxially with the injector main body 5. A portion of the plunger 9 is disposed inside the injector main body 5 via the rotary member 8, and the other portion of the plunger 9 is disposed protruding rearward from the rotary member 8.

The rod is disposed inside the hollow body formed by the injector main body 5 and the injection tube 7. The rod is connected to the plunger 9 and serves to advance the intraocular lens 4.

As shown in FIG. 2, the lens setting portion 11 includes a bottom surface portion 11a, lens receiving portions 11b, and lens guide portions 11c. The lens receiving portions 11b receive and support the intraocular lens 4 from below. The intraocular lens 4 is set on the lens setting portion 11 in a state in which the forward support portion 42a is disposed on the front side (the direction X1) as its name suggests, and the rearward support portion 42b is disposed on the rear side (the direction X2) as its name suggests. The intraocular lens injector 1 is of a preload type in which the intraocular lens 4 is set on the lens setting portion 11 of the injector main body 5 beforehand. Therefore, the intraocular lens 4 is one of the constituent elements of the intraocular lens injector 1. However, in carrying out the present invention, the intraocular lens injector 1 is not necessarily required to be of the preload type. The configuration of the intraocular lens injector 1 excluding a protrusion 20, which will be described later, can be expressed as follows. “An intraocular lens injector 1 including:
the hollow body which is an injector main body 5;
a lens setting portion 11 provided inside the hollow body;
an advancing member (a slider 6 or a rod connected to a plunger 9) for advancing the intraocular lens 4 disposed in the lens setting portion 11;
the protrusion 20 which is disposed forward of the lens setting portion 11 and protrudes from an inner wall of the hollow body; and
an injection tube 7 disposed at a front end portion of the hollow body,
the injection tube 7 having a hollow injection tube body 7a and a tubular nozzle portion 7b.”

### Configuration of Case

As the configuration of the case, for example, a configuration disclosed in Patent Document 1 to the applicant of the present invention may be adopted. With regard to the configuration of the case, the configuration disclosed in Patent Document 1 is adopted for the content that is not described below, and the description of Patent Document 1 is incorporated in the present specification in its entirety.

Note that, in case of any discrepancies between the content of WO 2018/003854 above and the content of Patent Document 1, the description in WO 2018/003854 takes precedence. For example, Patent Document 1 states that an intraocular lens 4 is set on a lens setting portion in a state in which a forward support portion is bent by a protrusion 80; however, in the present embodiment, as described in WO 2018/003854, a portion on which the intraocular lens 4 before being bent is placed is treated as the lens setting portion 11.

FIG. 3 is a perspective view of a case of the intraocular lens injector according to the embodiment of the present invention.

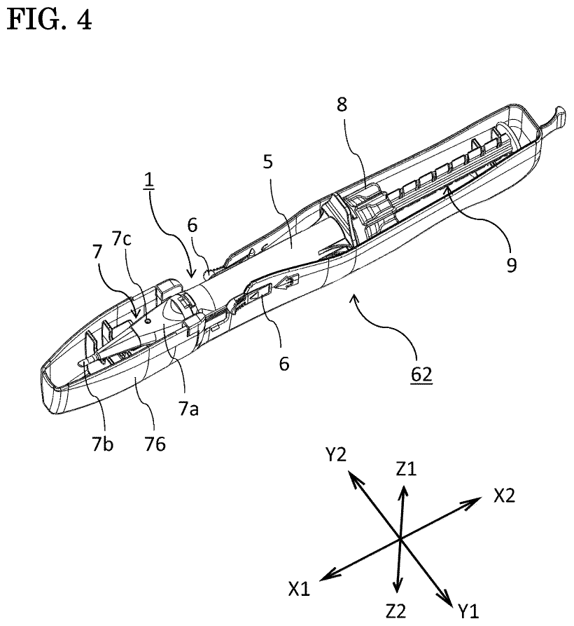

FIG. 4 is a perspective view showing a state in which the intraocular lens injector according to the embodiment of the present invention is attached to the case.

The intraocular lens injector 1 according to the present embodiment can be detachably fitted into a case 62 that is integrally molded from a synthetic resin material, for example. As shown in FIG. 10 of Patent Document 1 (corresponding to FIG. 3 of the present application), in the case 62, a U-shaped wall portion 76 extends perpendicularly from a bottom plate portion 75 so as to surround the intraocular lens injector 1, and the protrusion 20 extends perpendicularly from a predetermined position on the bottom plate portion 75.

As shown in FIG. 11 of Patent Document 1 (corresponding to FIG. 4 of the present application), the protrusion 20 is configured so as to be inserted into a protrusion insertion hole 73 of the intraocular lens injector 1 when the intraocular lens injector 1 is fitted into the case 62. Thus, in the intraocular lens injector 1, the protrusion 20 of the case 62 can be disposed in a central region of a space in which a leading end member 71 moves.

As shown in FIG. 4, in a state in which the intraocular lens injector 1 is fitted in the case 62, the intraocular lens 4 placed on the lens setting portion 11 is advanced toward a discharge hole of the nozzle portion 7b using the sliders 6. Note that, in the case where a configuration without the sliders 6 is adopted, the intraocular lens 4 may be advanced using the plunger 9 (directly by the rod connected to the plunger 9). For this reason, the sliders 6 or the rod connected to the plunger 9 is also referred to as an “advancing member”.

As a result of the advancement of the intraocular lens 4, the forward support portion 42a of the intraocular lens 4 abuts against the protrusion 20, and a leading end of the forward support portion 42a is bent rearward (in the direction X2), that is, in the opposite direction to the lens advancing direction (forward, the direction X1), and is consequently bent into a substantially U shape.

Furthermore, the intraocular lens injector 1 is removed from the case 62 in this state, the intraocular lens 4 with the forward support portion 42a still being bent is then gradually pushed forward (in the direction X1) by the plunger 9, and the intraocular lens 4 can thus be released from the nozzle portion 7b.

### Details of Protrusion According to the Present Embodiment

On the other hand, when releasing the intraocular lens 4, a problem arises in that, as described above, the leading end of the forward support portion 42a enters between an inner wall of the hollow body and an outer surface of the optical portion 41. To address this problem, in the present embodiment, the intraocular lens injector 1 with the protrusion 20 having the following form is used.

“An intraocular lens injector 1 for injecting an intraocular lens 4 into an eye, the intraocular lens 4 having an optical portion 41 and support portions 42a and 42b extending from the optical portion 41, the intraocular lens injector 1 including:
a hollow body having a lens setting portion 11 on which the intraocular lens 4 is set, with an optical axis direction of the intraocular lens 4 serving as a vertical direction; and
a protrusion 20 protruding from an inner wall of the hollow body, and having at least one step 22 (22a and 22b) directed upward when viewed from the lens setting portion 11 in a forward direction, and a wall 23a disposed forward of the step.”

The form of the protrusion 20 of the present embodiment will be described in detail. The movement of the forward support portion 42a when the intraocular lens 4 is being advanced using the sliders 6 from a state in which the intraocular lens 4 is placed on the lens setting portion 11 will be described in chronological order.

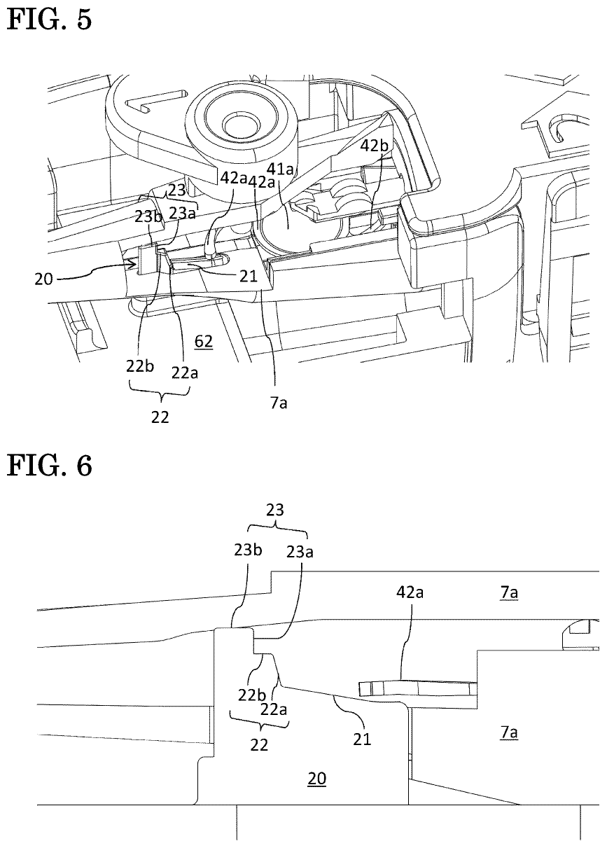

FIG. 5 is a perspective view showing a state of the protrusion and its vicinity in the intraocular lens injector according to the embodiment of the present invention.

FIG. 6 is a side view showing a state of the protrusion and its vicinity in the intraocular lens injector according to the embodiment of the present invention, when viewed in a direction from Y1 toward Y2.

FIG. 7 is a schematic cross-sectional view showing movements of the forward support portion (when viewed in cross section) relative to the protrusion in the intraocular lens injector according to the embodiment of the present invention.

When the intraocular lens 4 starts advancing, the forward support portion 42a abuts against a rising portion 22a of a step 22 of the protrusion 20. As shown in FIG. 6, this step 22 is a single step directed upward, or in other words, a single step extending upward and forward. As the intraocular lens 4 continues to advance, the forward support portion 42a is bent rearward due to abutting against the step 22, and the forward support portion 42a slides up the step 22 due to the advancement of the optical portion 41 ((a)→(b)→(c) in FIG. 7).

As the intraocular lens 4 further continues to advance, the forward support portion 42a slides all the way up to the top of the step 22. Once the forward support portion 42a has slid all the way to the top of the step 22, the forward support portion 42a starts advancing again toward a wall portion 23a located forward of the step 22 while moving along the top of the step 22 ((c)→(d) in FIG. 7). During the above-described sequential movements of moving along the top of the step 22 and advancing again, the bending of the forward support portion 42a is temporarily suppressed compared with that during the movement of sliding up the step 22.

Then, as the intraocular lens 4 further advances, the same phenomenon as that in the case of the first step 22 occurs, or more specifically, the forward support portion 42a slides up the wall 23a, and the degree of bending of the forward support portion 42a starts increasing again ((d)→(e) in FIG. 7).

In this manner, relative to the timing of advancement of the folded peripheral portion 41b of the optical portion 41 of the intraocular lens 4, the timing of movement of the leading end of the forward support portion 42a to the vicinity of the inner wall of the hollow body can be delayed. As a result, the accuracy of tacking can be improved.

Note that the protrusion 21 disclosed in Patent Document 1, which is the protrusion for bending the forward support portion, is a simple pin-like protrusion. Even if this pin-like protrusion can be regarded as a step, unlike the protrusion 20 of the present embodiment, a wall 23a does not exist forward of such a step. Furthermore, as for the other protrusions 52 and 80 disclosed in Patent Document 1, even though a concave curve is formed when they are viewed from the lens setting portion in the forward direction, there is only a single step when viewed from the lens setting portion in the forward direction, and unlike the protrusion 20 of the present embodiment, no wall 23a exists forward of the step.

There is no particular limitation on a specific form of the protrusion 20, as long as it satisfies the above-described configuration (i.e., protrudes from the inner wall of the hollow body and has the step and the wall). For example, the protrusion 20 may have at least two steps that protrude from bottom to top of the inner wall of the hollow body and are directed upward when viewed from the lens setting portion 11 in the forward direction.

As shown in FIGS. 5 and 6, when the protrusion 20 protrudes from bottom to top, there is no particular limitation on the number of steps directed upward as long as two or more steps are provided, but it is preferable that the number of steps is 2 to 4, because in this case the above-described timings can be effectively shifted, and the intraocular lens injector 1 can be easily produced. It goes without saying that, when even more steps are provided (or in other words, when the height of the rising portion 22a of each step 22 in the vertical direction decreases), it is still possible to delay the timing of movement of the leading end of the forward support portion 42a to the vicinity of the inner wall of the hollow body relative to the timing of advancement of the folded peripheral portion 41b of the optical portion 41 of the intraocular lens 4, compared with the protrusions disclosed in Patent Document 1.

The step 22 directed upward will be described in detail. For example, it is preferable that the rising portion 22a, which is the rearmost portion of the step, is disposed inclined at 45° to 95° with respect to the advancing direction of the intraocular lens 4, where the upper side is taken as the positive side and the lower side is taken as the negative side, and the inclination of a top portion 22b of the step 22 is set within a range of −30° to 30° with respect to the advancing direction of the intraocular lens 4. The reason for this is that, with this configuration, the above-described timings can be effectively shifted, and the accuracy of tacking is likely to be further improved. Similarly, it is preferable that the wall 23a (a rising portion of a second step 23), which is disposed forward of the step 22, is disposed inclined at 60° to 100° with respect to the advancing direction of the intraocular lens 4. Note that the inclination of a top portion 23b of the second step 23 is not particularly limited, but may be set within a range of −30° to 30° with respect to the advancing direction of the intraocular lens 4 as is the case with the top portion 22b of the first step 22. In the following description, the “step” refers to the first step 22 unless otherwise specified.

The height of the rising portion 22a, which is the rearmost portion of the step 22, in the vertical direction may be 0.3 to 1.5 mm, for example. The length of the top portion 22b of the step 22 in a front-rear direction may be 0.2 to 1.5 mm. The height of the wall 23 a in the vertical direction may be 0.3 to 1.5 mm, for example. When a plurality of steps are provided, the heights of the rising portions of the individual steps in the vertical direction may be uniformly set to 0.3 to 1.5 mm. When these conditions are satisfied, the above-described timings can be effectively shifted, and the accuracy of tacking is likely to be improved.

Note that, in consideration of the use as the intraocular lens injector 1, it is preferable that the overall height of the protrusion 20 in the vertical direction is 1.5 to 3.0 mm.

As shown in FIGS. 5 to 7, a slope (inclined surface) 21 extending upward and forward may be disposed rearward of the step 22. With the slope 21 disposed, when the intraocular lens 4 starts advancing, the forward support portion 42a can be guided to the rising portion 22a of the step 22 against which the forward support portion 42a should abut. Note that the angle of the slope 21 is not particularly limited, but is preferably more than 0° and 45° or less with respect to the advancing direction of the intraocular lens in order for the slope 21 to be a slope.

Note that the slope 21 is preferably continuous with the bottom of the inner wall of the hollow body, but a certain difference (e.g., 0.1 mm or less in the vertical direction) in level at the boundary therebetween is acceptable. If the difference in level is greater than an acceptable value, the slope 21 can be regarded as a step 22. In consideration of such a situation, the expression “at least two steps” is used in this specification.

FIG. 8 is a schematic side view showing a state of a protrusion and its vicinity in an intraocular lens injector according to a variation (first variation) of the present invention, when viewed in the direction from Y1 toward Y2.

Note that, although a case in which the step 22 and the wall 23a are integrated has been described by way of example in the present embodiment, the step 22 and the wall 23a may be formed spaced apart from each other as shown in FIG. 8. However, if the separation distance between the step 22 and the wall 23a is excessively large, the forward support portion 42a may fall into the gap therebetween, and tacking may thus be affected. For this reason, if the step 22 and the wall 23a are spaced apart from each other, it is preferable that the separation distance is smaller than the minimum cross-sectional width of the forward support portion 42a.

With regard to the form of the protrusion 20 protruding from the inner wall of the hollow body, in the present embodiment, a case in which, as disclosed in Patent Document 1, the protrusion 20 is provided to the case 62 and the protrusion insertion hole 73 is formed in the intraocular lens injector 1 has been described by way of example.

That is to say, “an intraocular lens injector 1, further including a case 62 configured to be fitted to the hollow body,
wherein the protrusion 20 is provided to the case 62 and a hole 73 is formed in the hollow body so that the protrusion 20 and the hole 73 can be fitted to each other” has been described by way of example in the present embodiment.

However, it goes without saying that the protrusion 20 or a portion thereof may be provided to the inner wall of the hollow body of the intraocular lens injector 1.

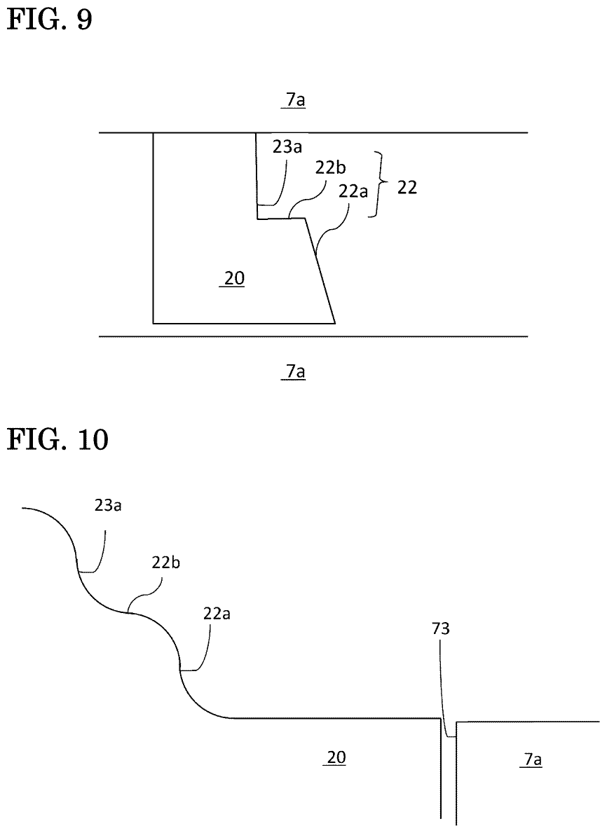

Furthermore, in the present embodiment, a case in which the protrusion 20 protrudes from the bottom to the top has been described by way of example. This case is preferred; however, the present invention is not limited to this case. FIG. 9 is a schematic side view showing a state of a protrusion and its vicinity in an intraocular lens injector according to a variation (second variation) of the present invention, when viewed in the direction from Y1 toward Y2.

For example, as shown in FIG. 9, in the case where the protrusion 20 protrudes from the top toward the bottom of the inner wall of the hollow body, the protrusion 20 may be disposed such that it protrudes to a position above the bottom of the inner wall and the step 22 directed upward and the wall 23a hang from the top of the inner wall of the hollow body when viewed from the lens setting portion 11 in the forward direction. Similarly, a configuration is also possible in which the protrusion 20 protrudes from a lateral side of the inner wall of the hollow body.

As a result of examination by the inventor of the present invention, it was found that the accuracy of tacking can be further improved by chamfering a part between the rising portion 22a, which is the rearmost portion, and the top portion 22b of the step 22, or in other words, a corner portion of the step. There is no particular limitation on the form of the chamfering, and the corner may be cut off to form a flat surface portion as shown in FIGS. 5 and 6, or may be rounded.

Furthermore, the chamfering described in the previous paragraph may be performed on all the corner portions of the steps 22 and 23.

FIG. 10 is a schematic side view showing a state of a protrusion and its vicinity in an intraocular lens injector according to a variation (third variation) of the present invention, when viewed in the direction from Y1 toward Y2.

In the case of FIG. 10, when tangents to curved surfaces of a portion of the rising portion 22a and a portion of the wall 23a are inclined at 60° to 100°, for example, with respect to the advancing direction of the intraocular lens 4, the bending and sliding-up of the forward support portion 42a can be effectively achieved. Furthermore, when the gradient of a tangent to a portion corresponding to the top portion 22b of the step 22 is −30° to 30°, for example, with respect to the advancing direction of the intraocular lens 4, the forward support portion 42a moves up onto and along the portion 22b, and the bending thereof is thus temporarily suppressed.

Moreover, as a result of examination by the inventor of the present invention, it was also found that the extent of the risk that the forward support portion 42a will enter between the outer surface of the optical portion 41 and the inner wall of the intraocular lens injector 1 varies depending on the conditions (in particular, flexibility of the peripheral portion 41b of the intraocular lens 4) of the surgical procedure. A configuration that reflects the above-described findings is as follows.

“An intraocular lens injector further including the intraocular lens 4,
wherein the optical portion 41 and a base end of each support portion 42a, 42b in the intraocular lens 4 are made of a soft material, and
the optical portion 41 of the intraocular lens 4 has an effective optical portion 41a exhibiting an optical function, and a flat peripheral portion 41b disposed around the effective optical portion 41a.”

The forms of the protrusion 20 according to the present embodiment that can be adopted have been specifically specified above. On the other hand, the technical idea of the present invention is to delay the timing of movement of the leading end of the forward support portion 42a to the vicinity of the inner wall of the intraocular lens injector 1, relative to the timing of advancement of the folded peripheral portion 41b of the optical portion 41 of the intraocular lens 4. The technical idea of the present invention can be expressed as a configuration below.

“An intraocular lens injector 1 for injecting an intraocular lens 4 into an eye, the intraocular lens 4 including an optical portion 41 having an optical function and support portions 42a and 42b extending from the optical portion 41, the intraocular lens injector 1 including:
a protrusion 20 for bending a forward support portion 42a while abutting against the forward support portion 42a, the forward support portion 42a being one of the support portions 42a and 42b and being disposed on the front side in an advancing direction of the intraocular lens 4, when making the intraocular lens 4 advance along an inner wall of a hollow body in which the intraocular lens 4 is set and thereby releasing the intraocular lens 4 from the hollow body in a state in which the intraocular lens 4 is folded with an inner surface of the optical portion 41 facing inward,
wherein the protrusion 20 has a retainer that temporarily suppresses progress of bending of the forward support portion 42a by retaining the forward support portion 42a, when the intraocular lens 4 is being folded.”

Note that the foregoing preferred examples in which the forms of the protrusion 20 according to the present embodiment that can be adopted have been specifically specified can be applied to this configuration as well.

The protrusion 20 in the previous paragraph corresponds to the step 22 and the wall 23a that have been described above. Also, the retainer included in this protrusion 20 corresponds to the top portion 22b of the step 22 and the wall 23a that have been described above. That is to say, the forward support portion 42a temporarily fits between the top portion 22b of the step 22 and the wall 23a and is thereby temporarily retained. However, the retention is only temporary, and as the intraocular lens 4 further advances, the same phenomenon as that in the case of the first step 22 occurs, or more specifically, the forward support portion 42a slides up the wall 23a, and the degree of bending of the forward support portion 42a starts increasing again. Note that, when the present invention is expressed as described above, the protrusion 20 is not limited to the steps 22 and 23. Also, the retainer is not limited to the top portion 22b of the step 22 and the wall 23a located forward thereof.

More preferably, the technical idea of the present invention can be expressed as a configuration below.

“An intraocular lens injector 1, wherein the protrusion 20 has a first abutting portion for bending the forward support portion 42a while abutting against the forward support portion 42a when the intraocular lens 4 is being advanced by the advancing member, and the retainer disposed forward of the first abutting portion, and
the retainer has a support receiving portion for supporting the forward support portion 42a after the forward support portion 42a is separated from the first abutting portion, and a second abutting portion which is disposed forward of the support receiving portion and abuts against the forward support portion 42a while the forward support portion 42a is supported by the support receiving portion.”

The first abutting portion, the support receiving portion, and the second abutting portion correspond to the following constituent elements respectively.

The first abutting portion corresponds to the rising portion 22a of the step 22 in FIGS. 5 to 10.

The support receiving portion corresponds to the top portion 22b of the step 22 in FIGS. 5 to 10.

The second abutting portion corresponds to the wall 23a that is disposed forward of the step 22 in FIGS. 5 to 10.

Note that the expression “a retainer that temporarily suppresses progress of bending” is used to mean the following.

The protrusion disclosed in Patent Document 1 is simply a single step when viewed in the advancing direction of the intraocular lens. When the forward support portion 42a abuts against a rising portion of this step, bending thereof progresses continuously. The inventor of the present invention found that, if the forward support portion 42a slides all the way up to the top of the step, the progress of bending is suppressed the moment the forward support portion 42a finishes sliding up; however, by that time, the leading end of the forward support portion 42a may have reached the inner wall of the hollow body earlier than the peripheral portion 41b of the optical portion 40 of the intraocular lens 4. In that case, the forward support portion 42a enters between the outer surface of the optical portion 40 that is the opposite surface to the inner surface thereof and the inner wall of the intraocular lens injector.

By contrast, in the present embodiment, since the second abutting portion is present forward of the step, the forward support portion 42a advances and abuts against the second abutting portion while being supported by the support receiving portion, and then the degree of bending of the forward support portion 42a starts increasing again. The expression “a retainer that temporarily suppresses progress of bending” is used in the above-described configuration in order to describe that the degree of bending of the forward support portion 42a increases, decreases, and increases in this manner. This means that at least two discontinuous changes in the degree of bending will result with an intervening decrease therebetween.

As described above, the specific forms and the configurations that express the technical idea of the present invention all make it possible to delay the timing of movement of the leading end of the forward support portion 42a to the vicinity of the inner wall of the hollow body, relative to the timing of advancement of the peripheral portion 41b of the folded intraocular lens. As a result, the accuracy of tacking can be improved.

### REFERENCE SIGNS LIST

1 Intraocular lens injector
5 Injector main body
6 Slider
7 Injection tube
7a Injection tube body
7b Nozzle portion
7c Injection hole
8 Rotary member
9 Plunger
11 Lens setting portion
11a Bottom surface portion
11b Lens receiving portion
11c Lens guide portion
73 Protrusion insertion hole
62 Case
20 Protrusion
21 Slope
22 (First) Step
22a Rising portion of (first) step
22b Top portion of (first) step
23 (Second) Step
23a Wall (of second step)
23b Top portion of (second) step
75 Bottom plate portion
76 U-shaped wall portion
4 Intraocular lens
41 Optical portion
41a Effective optical portion
41b Peripheral portion
42a Forward support portion
42b Rearward support portion

1-8. (canceled)

9. An intraocular lens injector for use with an intraocular lens including an optical portion that defines an optical axis and support portions extending from the optical portion, the intraocular lens injector comprising:
a hollow body defining a lens advancing direction and including an inner wall, a nozzle and a lens setting portion configured to hold the intraocular less such that the optical axis extends in an optical axis direction and one of the support portions defines a forward support portion; and
a protrusion, positioned between the nozzle and a lens setting portion such that the protrusion is contacted by the forward support portion as the intraocular lens moves in the lens advancing direction, and including
an inclined surface with a first slope having a component in the optical axis direction and a component in the lens advancing direction, a forward end, and a rearward end located at a rearmost end of the protrusion, and
a step having a rising portion, with a rearward end located at the forward end of the inclined surface and a second slope that is different than the first slope and that has at least a component in the optical axis direction, and a flat top portion.

10. The intraocular lens injector claimed in claim 9, wherein
the second slope that is greater than the first slope.

11. The intraocular lens injector claimed in claim 9, wherein
the protrusion includes a wall, with a third slope that has at least a component in the optical axis direction, located forwardly of the flat top portion of the step.

12. The intraocular lens injector claimed in claim 11, wherein
the third slope extends only in the optical axis direction.

13. The intraocular lens injector claimed in claim 9, wherein
the second slope extends only in the optical axis direction.

14. The intraocular lens injector claimed in claim 9, wherein
the hollow body comprises an injector main body having a front-end portion and an injection tube, having a hollow injection tube body and the nozzle, disposed at the front-end portion of the injector main body.

15. The intraocular lens injector claimed in claim 9, further comprising:
a lens advancing member for advancing the intraocular lens.

16. The intraocular lens injector claimed in claim 9, wherein
the step is a first step;
the protrusion includes a second step;
the first and second steps each have only a single respective rising portion and only a single respective flat top portion; and
the protrusion does not include additional steps.

17. The intraocular lens injector claimed in claim 9, further comprising:
the intraocular lens.

18. The intraocular lens injector claimed in claim 9, wherein
the step includes only a single rising portion and a single flat top portion.

19. The intraocular lens injector claimed in claim 9, wherein
the protrusion protrudes from the inner wall of the hollow body.

20. The intraocular lens injector claimed in claim 9, further comprising:
a case configured to partially surround the hollow body and to be secured to the hollow body;
wherein
the protrusion is located on the case;
the hollow body includes a protrusion hole that extends through the inner wall; and
the hollow body and the case are respectively configured such that the protrusion will extend through the protrusion hole when the case is secured to the hollow body and the intraocular lens injector is within the case.
